(19) United States
(12) Patent Application Publication
(10) Pub. No.: US 2008/0063707 A1
(43) Pub. Date: Mar. 13, 2008

(54) CONTROLLED RELEASE TABLET FORMULATIONS FOR THE PREVENTION OF ARRHYTHMIAS

(75) Inventors: Gregory N. Beatch, Vancouver (CA); Alan M. Ezrin, Miami, FL (US)

Correspondence Address: SEED INTELLECTUAL PROPERTY LAW GROUP PLLC, 701 FIFTH AVE, SUITE 5400, SEATTLE, WA, 98104, US

(73) Assignee: CARDIOME PHARMA CORP., Vancouver (CA)

(21) Appl. No.: 11/832,580

(22) Filed: Aug. 1, 2007

Related U.S. Application Data
(63) Continuation-in-part of application No. 10/838,470, filed on May 3, 2004.
(63) Continuation-in-part of application No. PCT/US04/13731, filed on May 3, 2004.
(60) Provisional application No. 60/467,159, filed on May 2, 2003.
(60) Provisional application No. 60/493,392, filed on Aug. 7, 2003.
(60) Provisional application No. 60/516,248, filed on Oct. 31, 2003.
(60) Provisional application No. 60/516,486, filed on Oct. 31, 2003.
(60) Provisional application No. 60/526,911, filed on Dec. 3, 2003.
(60) Provisional application No. 60/527,169, filed on Dec. 4, 2003.
(60) Provisional application No. 60/528,251, filed on Dec. 8, 2003.
(60) Provisional application No. 60/559,405, filed on Apr. 1, 2004.
(60) Provisional application No. 60/544,941, filed on Feb. 13, 2004.
(60) Provisional application No. 60/467,159, filed on May 2, 2003.
(60) Provisional application No. 60/493,392, filed on Aug. 7, 2003.
(60) Provisional application No. 60/516,248, filed on Oct. 31, 2003.
(60) Provisional application No. 60/516,486, filed on Oct. 31, 2003.
(60) Provisional application No. 60/526,911, filed on Dec. 3, 2003.
(60) Provisional application No. 60/527,169, filed on Dec. 4, 2003.
(60) Provisional application No. 60/528,251, filed on Dec. 8, 2003.
(60) Provisional application No. 60/559,405, filed on Apr. 1, 2004.
(60) Provisional application No. 60/544,941, filed on Feb. 13, 2004.
(60) Provisional application No. 60/916,129, filed on May 4, 2007.

(51) Int. Cl. A61K 31/402; A61K 9/22; A61P 9/06

(52) U.S. Cl. 424/465; 514/424

(57) ABSTRACT

Controlled release tablet formulations comprising a therapeutically effective amount of an ion channel modulating compound, or a pharmaceutically acceptable salt thereof, and one or more pharmaceutically acceptable excipients suitable for controlled release formulations are disclosed.

### CROSS REFERENCE TO RELATED APPLICATIONS

This application is a continuation-in-part of U.S. patent application Ser. No. 10/838,470 filed May 3, 2004 (currently pending), which claims the benefit under 35 U.S.C. §119(e) of U.S. Provisional Patent Application Nos. 60/467,159, filed May 2, 2003; 60/493,392, filed Aug. 7, 2003; 60/516,248, filed Oct. 31, 2003; 60/516,486, filed Oct. 31, 2003; 60/526,911, filed Dec. 3, 2003; 60/527,169, filed Dec. 4, 2003; 60/528,251, filed Dec. 8, 2003; 60/559,405, filed Apr. 1, 2004; and 60/544,941, filed Feb. 13, 2004;

This application is also a continuation-in-part of International Application No. PCT/US04/013731, accorded an International Filing Date of May 3, 2004, which claims the benefit under 35 U.S.C. §119(e) of U.S. Provisional Patent Application Nos. 60/467,159, filed May 2, 2003; 60/493,392, filed Aug. 7, 2003; 60/516,248, filed Oct. 31, 2003; 60/516,486, filed Oct. 31, 2003; 60/526,911, filed Dec. 3, 2003; 60/527,169, filed Dec. 4, 2003; 60/528,251, filed Dec. 8, 2003; 60/559,405, filed Apr. 1, 2004; and 60/544,941, filed Feb. 13, 2004.

This application also claims the benefit under 35 U.S.C. §119(e) of U.S. Provisional Patent Application No. 60/916,129, filed May 4, 2007.

The above-mentioned applications are incorporated herein by reference in their entireties.

### FIELD OF THE INVENTION

The present invention is directed to controlled release tablet formulations of ion channel modulating compound or pharmaceutically acceptable salts thereof. These controlled release tablet formulations are useful in preventing arrhythmia and other diseases, in particular atrial fibrillation, from occurring in mammals, preferably in humans.

### BACKGROUND OF THE INVENTION

Arrhythmias are abnormal rhythms of the heart. The term “arrhythmia” refers to a deviation from the normal sequence of initiation and conduction of electrical impulses that cause the heart to beat. Arrhythmias may occur in the atria or the ventricles. Atrial arrhythmias are widespread and relatively benign, although they place the subject at a higher risk of stroke and heart failure. Ventricular arrhythmias are typically less common, but very often fatal.

Atrial fibrillation is the most common arrhythmia encountered in clinical practice. It has been estimated that 2.2 million individuals in the United States have paroxysmal or persistent atrial fibrillation. The prevalence of atrial fibrillation is estimated at 0.4% of the general population, and increases with age. Atrial fibrillation is usually associate with age and general physical condition, rather than with a specific cardiac event, as is often the case with ventricular arrhythmia. While not directly life threatening, atrial arrhythmias can cause discomfort and can lead to stroke or congestive heart failure, and increase overall morbidity.

There are two general therapeutic strategies used in treating subjects with atrial fibrillation. One strategy is to allow the atrial fibrillation to continue and to control the ventricular response rate by slowing the conduction through the atrioventricular (AV) node with digoxin, calcium channel blockers or beta-blockers; this is referred to as rate control. The other strategy, known as rhythm control, seeks to convert the atrial fibrillation and then maintain normal sinus rhythm, thus attempting to avoid the morbidity associated with chronic atrial fibrillation. The main disadvantage of the rhythm control strategy is related to the toxicities and proarrhythmic potential of the anti-arrhythic drugs used in this strategy. Most drugs currently used to prevent atrial or ventricular arrhythmias have effects on the entire heart muscle, including both healthy and damaged tissue. These drugs, which globally block ion channels in the heart, have long been associated with life-threatening ventricular arrhythmia, leading to increased, rather than decreased, mortality in broad subject populations. There is therefore a long recognized need for antiarrhythmic drugs that are more selective for the tissue responsible for the arrhythmia, leaving the rest of the heart to function normally. Such drugs are less likely to cause ventricular arrhythmias.

Ion channel modulating compounds selective for the tissue responsible for arrhythmia are described in U.S. Pat. No. 7,057,053. Of particular interest to the present invention is the ion channel modulating compound known as vernakalant hydrochloride. Vernakalant hydrochloride is the non-proprietary name adopted by the United States Adopted Name (USAN) council for the ion channel modulating compound having the following formula: 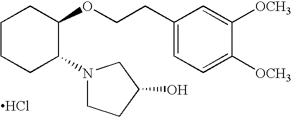
and a chemical name of (3R)-1-[(1R,2R)-2-(3,4-dimethoxyphenyl)ethoxy]cyclohexyl]pyrrolidin-3-ol hydrochloride. Vernakalant hydrochloride modifies atrial electrical activity through a combination of concentration-, voltage- and frequency-dependent blockade of sodium channels and blockade of ultra-rapidly activating (I_{Kur}) and transient outward (Iₜₒ) potassium channels. These combined effects prolong atrial refractoriness and rate-dependently slow atrial conduction. This unique profile provides an effective anti-fibrillatory approach expected to be suitable for conversion of atrial fibrillation and the prevention of recurrence of atrial fibrillation.

There therefore exists a need for controlled release tablet formulations of vernakalant hydrochloride for the prevention of the recurrence of arrhythmia in mammals, preferably in humans.

### SUMMARY OF THE INVENTION

The present invention is directed to controlled release tablet formulations for ion channel modulating compounds or pharmaceutically acceptable salts thereof. These controlled release tablet formulations are useful in the prevention of the recurrence of arrhythmia, particularly, atrial fibrillation and/or atrial flutter, in a mammal, preferably in a human, upon oral administration thereof to the mammal.

Accordingly, in one aspect this invention provides controlled release tablet formulations comprising a therapeutically effective amount of an ion channel modulating compound and one or more pharmaceutically acceptable excipients.

In another aspect, this invention provides controlled release tablet formulations comprising a therapeutically effective amount of vernakalant hydrochloride and one or more pharmaceutically acceptable excipients.

In another aspect, this invention provides controlled release tablet formulations comprising a therapeutically effective amount of vernakalant hydrochloride and one or more pharmaceutically acceptable excipients wherein at least one of the pharmaceutically acceptable excipients comprises a hydrophilic matrix polymer.

In another aspect, this invention provides controlled release tablet formulations comprising a therapeutically effective amount of vernakalant hydrochloride and one or more pharmaceutically acceptable excipients wherein at least one of the pharmaceutically acceptable excipients comprises an erodable retardant.

In another aspect, this invention provides controlled release tablet formulations comprising a therapeutically effective amount of vernakalant hydrochloride and one or more pharmaceutically acceptable excipients wherein at least one of the pharmaceutically acceptable excipients comprises a hydrophobic matrix polymer.

In another aspect, this invention provides controlled release tablet formulations comprising a therapeutically effective amount of vernakalant hydrochloride and one or more pharmaceutically acceptable excipients wherein one of the pharmaceutically acceptable excipients comprises a hydrophobic matrix polymer and another of the pharmaceutically acceptable excipients comprises a hydrophilic matrix polymer.

In another aspect, this invention provides for controlled release tablet formulations comprising 100 mg of an ion channel modulating compound, or a pharmaceutically acceptable salt thereof, and one or more pharmaceutically acceptable excipients.

In another aspect, this invention provides for controlled release tablet formulations comprising 100 mg of vernakalant hydrochloride and one or more pharmaceutically acceptable excipients.

In another aspect, this invention provides for controlled release tablet formulations comprising 100 mg of vernakalant hydrochloride and one or more pharmaceutically acceptable excipients wherein at least one pharmaceutically acceptable excipient comprises a hydrophilic matrix polymer.

In another aspect, this invention provides for controlled release tablet formulations comprising 300 mg of an ion channel modulating compound, or a pharmaceutically acceptable salt thereof, and one or more pharmaceutically acceptable excipients.

In another aspect, this invention provides for controlled release tablet formulations comprising 300 mg of vernakalant hydrochloride and one or more pharmaceutically acceptable excipients.

In another aspect, this invention provides for controlled release tablet formulations comprising 300 mg of vernakalant hydrochloride and one or more pharmaceutically acceptable excipients wherein at least one pharmaceutically acceptable excipient comprises a hydrophilic matrix polymer.

Preferably in this aspect, the hydrophilic matrix polymer is selected from the group consisting of carbomer, maltodextrin, hydroxyethyl cellulose, hydroxypropyl cellulose, hydroxypropyl methyl cellulose, and polyoxoacetate. More preferably, the hydrophilic matrix polymer is hydroxypropyl methyl cellulose.

In another aspect, this invention provides for controlled release table formulations comprising 300 mg of vernakalant hydrochloride and one or more pharmaceutically acceptable excipients wherein at least one pharmaceutically acceptable excipient comprises an erodable retardant.

Preferably in this aspect, the erodable retardant is selected from from the group consisting of cetyl alcohol or cetostearyl alcohol. More preferably, the erodable retardant is cetostearyl alcohol.

In another aspect, this invention provides for a method of preventing the recurrence of an arrhythmia in a mammal that has previously undergone one or more arrhythmia, wherein the method comprises administering to the mammal in need thereof a therapeutically effective amount of a controlled release formulation of the invention. Preferably, the arrhythmia is atrial fibrillation and/or atrial flutter.

### BRIEF DESCRIPTION OF THE FIGURE

FIG. 1 shows the dissolution profile of a comparative immediate release tablet formulation comprising 100 mg of the active ingredient over time.

### DETAILED DESCRIPTION OF THE INVENTION

This invention is directed to controlled release tablet formulations comprising a therapeutically effective amount of ion channel modulating compound, or a pharmaceutically acceptable salt thereof, and one or more pharmaceutically acceptable excipients. In particular, this invention is directed to controlled release tablet formulations comprising a therapeutically effective amount of vernakalant hydrochloride and one or more pharmaceutically acceptable excipients suitable for controlled release formulations, which, upon oral administration thereto, are effective in preventing the recurrence of arrhythmia in mammals, preferably in humans.

Accordingly, the controlled release tablet formulations of the invention are intended to be administered to a mammal, preferably a human, that has previously undergone one or more arrhythmias.

As used herein, unless the context makes clear otherwise, “prevention,” and similar words such as “prevented,” “preventing”, etc, preferably means keeping a subsequent arrhythmia from occurring in a mammal that has previously undergone one or more arrhythmias. Prevention, as used herein, may also mean a lessening of the severity of a subsequent arrhythmia if a subsequent arrhythmia does occur. Prevention, as used herein, may also mean postponing the time for onset of the subsequent arrhythmia. Prevention, as used herein may also mean lessening the probability that the mammal that has previously undergone one or more arrhythmias will suffer from a subsequent arrhythmia.

In one version of the invention, the arrhythmia to be prevented is atrial fibrillation. In another version, the arrhythmia to be prevented is atrial flutter.

Generally, the subject in which arrhythmia is be prevented is any mammal. In one version, the subject is a human. In another version, the subject is any domestic animal, including, but not limited to cats, dogs, etc. In another version, the subject is any farm animal, including, but not limited to pigs, cows, horses, etc.

As set forth above in the Summary of the Invention, the controlled release tablet formulations of the invention comprise a therapeutically effective amount of an ion channel modulating compound, or a pharmaceutically acceptable salt thereof, as the active ingredient and one or more pharmaceutically acceptable excipients. As used herein, a “therapeutically effective amount” refers to that amount of active ingredient sufficient to effect the desired prevention of arrhythmia in the mammal to which a formulation of the invention has been administered. As used herein, “pharmaceutically acceptable” refers to those compounds, salts, excipients and compositions, which are, within the scope of sound medical judgment, suitable for use in contact with the tissues of mammals, preferably humans, without excessive toxicity, irritation, allergic response, or other problem or complication, commensurate with a reasonable benefit/risk ratio. As used herein, and described in more detail below, a “pharmaceutically acceptable excipient” can be any pharmaceutically acceptable material, composition, or vehicle suitable for allowing the active ingredient to be released from the formulation in a controlled manner, including but not limited to, a liquid or solid filler, diluent, excipient, solvent or encapsulating material, which is involved in carrying or transporting the active ingredient to an organ, or portion of the body. Each pharmaceutically acceptable excipient must be compatible with the other ingredients of the formulation. Some examples of materials which can serve as pharmaceutically acceptable excipients include, but are not limited to, sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances routinely employed in pharmaceutical formulations and any substance identified herein as a pharmaceutically acceptable excipient.

As used herein, “controlled release” refers to the release of the active ingredient from the formulation in a sustained and regulated manner over a longer period of time than an immediate release formulation containing the same amount of the active ingredient would release during the same time period. For example, an immediate release formulation comprising vernakalant hydrochloride may release 80% of the active ingredient from the formulation within 15 minutes of administration to a human subject, whereas an extended release formulation of the invention comprising the same amount of vernakalant hydrochloride would release 80% of the active ingredient within a period of time longer than 15 minutes, preferably within 6 to 12 hours. Controlled release formulations allows for less frequency of dosing to the mammal in need thereof. In addition, controlled release formulations may improve the pharmacokinetic or toxicity profile of the compound upon administration to the mammal in need thereof.

Active Ingredient

The ion channel modulating compounds, or pharmaceutically acceptable salts thereof, utilized in the formulations of the invention can be any ion channel modulating compound or pharmaceutical acceptable salt thereof. Preferably, the ion channel modulating compound is a compound described in U.S. Pat. No. 7,057,053. More preferably, the ion channel modulating compound is vernakalant hydrochloride. Vernakalant hydrochloride has been shown to be orally bioavailable in humans and animals (e.g., in dogs). The compound is rapidly absorbed, and has a linear pharmacokinetic profile in humans following a 10-minute infusion. The half-life of the compound in healthy volunteers has been shown to be approximately 2 hours compared to 3-4 hours in patients with recent onset atrial fibrillation.

Vernakalant hydrochloride is highly soluble in citrate solution (143 mg/mL), and has a pH of 3.2 in water, and a pKa of 8.32. It is anhydrous, and is stable under long term and accelerated conditions.

Excipients

Exemplary excipients that are suitable for the controlled release formulations of the invention are listed in Tables 1 to 5 below, along with their chemical/ brand name, compendial status and function.

TABLE 1
EXCIPIENTS FOR A CONTROLLED RELEASE FORMULATION
UTILIZING A HYDROPHILIC MATRIX SYSTEM

| | Compendial | |
|---|---|---|
| Excipient | Status | Function |
| Avicel (Microcrystalline | USP/EP/JP | Filler/Diluent |
| Cellulose) | | |
| Carbopol 971 P or | USP-NF | Hydrophilic matrix |
| Carbopol 71 G | | system polymer |
| (Carbomer) | | |
| Colloidal Silicon Dioxide | USP/EP/JP | Glidant |
| Emcompress (Calcium | USP-NF | Filler/Diluent |
| Phosphate Dibasic) | | |
| Glucidex 9 (Maltodextrin) | USP-NF/EP | Hydrophilic matrix |
| | | system polymer |
| Hydroxyethyl Cellulose | USP/EP/JP | Hydrophilic matrix |
| | | system polymer |
| Hydroxypropyl Cellulose | USP/EP/JP | Hydrophilic matrix |
| | | system polymer |
| Lactose Fast Flo (Lactose | USP/EP/JP | Filler/Diluent |
| Monohydrate) | | |
| Magnesium Stearate | USP/EP/JP | Lubricant |
| (Non-Bovine) | | |
| Methocel E4M Premium | USA | Hydrophobic polymer |
| (Hydroxypropyl methyl | | |
| cellulose, controlled | | |
| release grade) | | |
| Methocel K4M | USP | Hydrophilic matrix |
| (Hydroxypropyl methyl | | system polymer |
| cellulose, controlled | | |
| release grade) | | |
| Polyox WSR 1105 | House | Hydrophilic matrix |
| (Polyoxoacetate) | | system polymer |
| Polyvinyl pyrrolidone | USP | Hydrophilic matrix |
| | | system |
| | | polymer/Binder |
| Prosolv SMCC 90 | USP-NF/EP | Filler/Glidant |
| (Silicified microcrystalline | | |
| cellulose) | | |
| Sodium carboxymethyl | USP | Hydrophobic polymer |
| cellulose | | |
| Starch 1500 | USP-NF | Binder for wet |
| (Pregelatinized starch) | | densification |
| Stearic Acid | USP/EP | Lubricant |

TABLE 2
EXCIPIENTS FOR A CONTROLLED RELEASE FORMULATION
UTILIZING A HYDROPHOBIC MATRIX SYSTEM

| | Compendial | |
|---|---|---|
| Excipient | Status | Function |
| Avicel (Microcrystalline | USP/EP/JP | Filler/Diluent |
| Cellulose) | | |
| Colloidal Silicon Dioxide | USP/EP/JP | Glidant |
| Emcompress (Calcium | USP-NF | Filler/Diluent |
| Phosphate Dibasic) | | |
| Ethocel STD-4 (Ethyl | USP-NF | Hydrophobic matrix |
| Cellulose) | | system polymer |
| Eudragit RSPO | USP-NF | Hydrophobic matrix |
| (Methacrylic acid | | system polymer |
| copolymer) | | |
| Eudragit S-100 | USP-NF | Hydrophobic matrix |
| (Methacrylic acid | | system polymer r |
| copolymer | | |
| Kollidon SR (Polyvinyl | USP-NF | Hydrophobic matrix |
| acetate pyrrolidone) | | system polymer |
| Lactose Fast Flo (Lactose | USP/EP/JP | Filler/Diluent |
| monohydrate) | | |
| Magnesium Stearate | USP/EP/JP | Lubricant |
| (Non-Bovine) | | |
| Plasdone K29/32 | USP-NF | Binder |
| (Povidone, polyvinyl | | |
| pyrrolidone) | | |
| Polyethylene Glycol 8000 | USP-NF | Hydrophobic matrix |
| (Polyethylene glycol) | | system polymer |
| Stearic Acid | USP/EP | Lubricant |

TABLE 3
EXCIPIENTS FOR A CONTROLLED RELEASE FORMULATION
UTILIZING A FAT-WAX SYSTEM

| | Compendial | |
|---|---|---|
| Excipient | Status | Function |
| Avicel (Microcrystalline | USP/EP/JP | Filler/Diluent |
| cellulose) | | |
| Colloidal Silicon Dioxide | USP/EP/JP | Glidant |
| Emcompress (Calcium | USP-NF | Filler/Diluent |
| phosphate dibasic) | | |
| Kalcol 6098 (Cetyl | USP | Erodable Retardant |
| alcohol) | | |
| Kalcol 6850P | USN | Erodable Retardant |
| (Cetostearyl alcohol) | | |
| Lactose Fast Flo (Lactose | USP/EP/JP | Filler/Diluent |
| Monohydrate) | | |
| Magnesium Stearate | USP/EP/JP | Lubricant |
| (Non-Bovine) | | |
| Prosolv SMCC 90 | USP-NF/EP | Filler |
| (Silicified microcrystalline | | |
| cellulose) | | |
| Stearic Acid | USP/EP | Lubricant |

TABLE 4
EXCIPIENTS FOR A CONTROLLED RELEASE FORMULATION
UTILIZING A HYDROPHILIC/HYDROPHOBIC MATRIX SYSTEM

| | Compendial | |
|---|---|---|
| Excipient | Status | Function |
| Ethocel (Ethyl Cellulose) | USP-NF | Hydrophobic matrix |
| | | system polymer |
| Eudragit RSPO | USP-NF | Hydrophobic matrix |
| (Methacrylic acid | | system polymer |
| copolymer) | | |
| Eudragit S-100 | USP-NF | Hydrophobic matrix |
| (Methacrylic acid | | system polymer |
| copolymer) | | |
| Kollidon SR (Polyvinyl | USP-NF | Hydrophobic matrix |
| acetate) | | system polymer |
| Methocel E4M Premium | USP | Hydrophobic matrix |
| (Hydroxypropyl methyl | | system polymer |
| cellulose, controlled | | |
| release grade) | | |
| Methocel K4M | USP | Hydrophilic matrix |
| (Hydroxypropyl methyl | | system polymer |
| cellulose, controlled | | |
| release grade) | | |
| Polyvinyl pyrrolidone | USP | Hydrophilic matrix |
| | | system polymer/Binder |
| Lactose Fast Flo (Lactose | USP/EP/JP | Filler/Diluent |
| monohydrate) | | |
| Avicel (Microcrystalline | USP/EP/JP | Filler/Diluent |
| cellulose) | | |
| Emcompress (Calcium | USP-NF | Filler/Diluent |
| Phosphate Dibasic) | | |
| Colloidal Silicon Dioxide | USP/EP/JP | Glidant |
| Magnesium Stearate | USP/EP/JP | Lubricant |
| (Non-Bovine) | | |
| Stearic Acid | USP/EP | Lubricant |

TABLE 5
EXCIPIENTS FOR A CONTROLLED RELEASE FORMULATION
UTILIZING A FILM-COATED PARTICULATE SYSTEM

| | Compendial | |
|---|---|---|
| Excipient | Status | Function |
| Ac-Di-Sol (Sodium | USP-NF | Disintegrant |
| crosscarmellose) | | |
| Avicel (Microcrystalline | USP/EP/JP | Filler/Diluent |
| Cellulose) | | |
| Colloidal Silicon Dioxide | USP/EP/JP | Glidant |
| Emcompress (Calcium | USP-NF | Filler/Diluent |
| Phosphate Dibasic) | | |
| Explotab (Sodium Starch | USP-NF | Disintegrant |
| Glycolate) | | |
| Lactose Fast Flo (Lactose | USP/EP/JP | Filler/Diluent |
| monohydrate) | | |
| Magnesium Stearate | USP/EP/JP | Lubricant |
| (Non-Bovine) | | |
| Plasdone K29/32 | USP | Binder |
| (Povidone, polyvinyl | | |
| pyrrolidone) | | |
| Starch 1500 (Pre- | USP-NF | Glidant/Disintegrant |
| gelatinized Starch) | | |
| Stearic Acid | USP/EP | Lubricant |

Exemplary excipients suitable for immediate release tablet formulations are listed in the following Table 6, along with their chemical/brand name, compendial status and function.

TABLE 6
EXCIPIENTS FOR IMMEDIATE RELEASE FORMULATIONS

| Excipient | Compendial Status | Function |
|---|---|---|
| Avicel (Microcrystalline | USP/EP/JP | Filler/Diluent |
| Cellulose) | | |
| Colloidal Silicon Dioxide | USP/EP/JP | Glidant |
| Emcompress (Calcium | USP-NF | Filler/Diluent |
| Phosphate Dibasic) | | |
| Lactose Fast Flo (Lactose | USP/EP/JP | Filler/Diluent |
| monohydrate) | | |
| Magnesium stearate | USP/EP/JP | Lubricant |
| (Non-Bovine) | | |
| Polyvinyl pyrrolidone | USP | Binder |
| Silicified Microcrystalline | USP | Filler/Diluent |
| Cellulose | | |
| Sodium Starch Glycolate | USP | Disintegrant |
| Starch (Pre-gelatinized) | USP | Glidant/Disintegrant |
| Stearic Acid | USP/EP | Lubricant |

### Preparation of the Formulations of the Invention

The controlled release tablet formulations of the invention are formulated so that a final dosage form exhibits many desirable properties including, but not limited to, good tabletting characteristics (e.g., good flow, compression, appearance, weight variation, hardness, friability, content uniformity and dissolution rate properties), good bioavailability profiles (e.g., greater than 6 hours in vivo active ingredient release profile for a controlled release formulation of the invention), excellent stress and long-term stability, small tablet size, and simple, but efficient, and cost-effective manufacturing.

Controlled release tablet formulations of the invention may be made by incorporating the ion channel modulating compound, or its pharmaceutically effective salt, (collectively referred to herein as the “active ingredient”), preferably vernakalant hydrochloride, within a matrix system, including, but not limited to, a hydrophilic matrix system, a hydrophilic non-cellulose matrix system, a hydrophobic (plastic matrix system), or a hydrophilic/hydrophobic matrix system; within a fat-wax system; or within a film-coated particulate system.

Hydrophilic matrix systems show uniform and constant diffusion of the active ingredient from a tablet prepared with a hydrophilic, gelling polymer (i.e., a hydrophilic matrix system polymer) after the tablet is placed in an aqueous environment. Release of the active ingredient from the system is controlled by a gel diffusional barrier which is formed by a process that is usually a combination of gel hydration, diffusion of the active ingredient, and gel erosion.

Hydrophobic (plastic) matrix systems utilize inert, insoluble polymers (i.e., hydrophobic matrix system polymers) and copolymers to form a porous skeletal structure in which the active ingredient is embedded. Controlled release is effected by diffusion of the active ingredient through the capillary wetting channels and pores of the matrix, and by erosion of the matrix itself.

Hydrophilic/hydrophobic matrix systems utilize a combination of hydrophilic and hydrophobic polymers that forms a soluble/insoluble matrix in which the active ingredient is embedded. Controlled release of the active ingredient is by pore and gel diffusion as well as tablet matrix erosion. The hydrophilic polymer is expected to delay the rate of gel diffusion.

In fat-wax systems, the active ingredient is incorporated in a hot melt of a fat-wax (i.e., erodable retardant matrix), solidified, sized and compressed with appropriate tablet excipients. Controlled release of the active ingredient is effected by pore diffusion and erosion of the fat-wax system. The addition of a surfactant as a wicking agent helps water penetration of the system to cause erosion.

Film-coated particulate systems include time-release granulations, prepared by extrusion-spheronization process or by conventional granulation process that have been film-coated to produce differing species of controlled release particles with specific active ingredient release characteristics. Controlled release particles may be compressed together with appropriate excipients to produce tablets with the desired controlled release profile. The release of the active ingredient is by particle erosion in either acid (gastric) or alkaline (intestinal) pH.

Immediate release tablet formulations comprising the active ingredient were prepared for comparison purposes only by compounding the active ingredient with appropriate excipients, including, but not limited to, immediate release fillers, binders, glidants, disintegrants and lubricants, to give satisfactory tabletting characteristics and subsequent rapid disintegration and dissolution of the tablets.

Controlled release tablet formulations of the invention may be manufactured by methods including, but not limited to, direct compression (dry blending the active ingredient with flowable excipients, followed by compression), wet granulation (application of a binder solution to powder blend, followed by drying, sizing, blending and compression), dry granulation or compaction (densifying the active ingredient or active ingredient/powder blend through slugging or a compactor to obtain flowable, compressible granules), fat-wax (hot melt) granulation (embedding the active ingredient in molten fatty alcohols, followed by cooling, sizing, blending and compression), and film-coating of particulates (dry blend, wet granulation, kneading, extrusion, spheronization, drying, film-coating, followed by blending of different species of film-coated spheres, and compression).

Of particular interest to the invention is the method of manufacturing controlled release tablet formulations of the invention such that each tablet comprise 100 mg or 300 mg of the active ingredient. The methods for manufacturing these tablets include, but are not limited, to the following methods:

a. Direct compression.

b. Wet densification of the active ingredient and Starch 1500 or Povidone K29/32 with purified water, followed by tray drying to a moisture level of 2-3% w/w/and blending with direct compression excipients.

c. Fat-wax (hot melt).

In one version of the direct compression method, the desired amount of the active ingredient and the desired amount of Starch 1500, Povidone K29/32, Lactose Fast Flo, Anhydrous Emcompress or Carbopol 71G are mixed by hand in a small polyethylene (PE) bag or a 500 mL high density polyethylene (HDPE) container for approximately one minute and then passed through a #30 mesh screen. The resulting blend is then mixed with the desired amounts of the remaining excipients in the desired formulation, excluding magnesium stearate and stearic acid, for approximately 2 minutes in either a small PE bag or a 500 mL HDPE container. Approximately 1 g of the resulting mixture is then mixed with the desired amount of magnesium stearate and stearic acid, passed through a #30 mesh screen, added back to the remaining resulting mixture and then blended for approximately one minute. The resulting blend is then compressed into tablets at a final tablet weight of 225 mg or 300 mg (for tablets containing 100 mg active ingredient) or 630 mg or 675 mg (for tablets containing 300 mg active ingredient using a conventional bench top tablet press.

In another version of the direct compression method, the desired amount of the pre-screened (#40 mesh) active ingredient and Starch 1500 are placed in a 4 quart V-shell and blended at 25 rpm for 3 minutes. To the resulting blend is added the desired amounts of pre-screened (#30 mesh) Prosolv SMCC 90, Lactose Fast Flow, Methocel K4M and stearic acid (pre-screened through a #40 mesh) and the resulting mixture is mixed for 5 minutes at 25 rpm. Magnesium stearate is then added to an equal amount of the resulting mixture, which is then blended in a small polyethylene bag for approximately 1 minute, passed through a #30 mesh screen by hand and returned to the resulting mixture. The final resulting mixture is blended for 2 minutes at 25 rpm and then compressed into tablets at a final tablet weight of 630 mg or 675 mg (for tablets containing 300 mg active ingredient) using a conventional tablet press.

In a version of the wet densification method, the desired amount of the active ingredient is mixed with the desired amount of Starch 1500 or Povidone K29/32 and the resulting mixture is passed through a #30 mesh screen. Purified water is added to the screened mixture until it reaches a satisfactory densification end point. The resulting wet mass is passed through a #12 mesh screen onto a tray and dried at 60° C. for 2 to 3 hours until a moisture level of 2-3% w/w is obtained. The resulting dry granules are passed through a #20 mesh screen into either a small PE bag or a 500 mL HDPE container. To the screened dry granules is added the desired amounts of the remaining excipients of the formulation, excluding magnesium stearate and stearic acid. The contents are mixed for approximately 2 minutes. Approximately 1 g of the resulting mixture is then mixed with the desired amounts of magnesium stearate and stearic acid, passed through a #30 mesh screen, added back to the remaining resulting mixture and then blended for approximately 1 minute. The final resulting blend is compressed into tables at a final tablet weight of 225 mg or 300 mg (for tablets containing 100 mg active ingredient) and 630 mg or 675 mg (for tablets containing 300 mg active ingredient) using a conventional tablet press.

In a version of the fat-wax (hot melt) method, the desired amount of fat-wax, preferably an erodable retardant selected from cetostearyl alcohol and cetyl alcohol, is placed in a stainless steel container, which is then heated on until the wax completely liquifies (i.e., completely melts). The desired amounts of the active ingredient, Lactose Fast Flo and Prosolv SMCC90 are then added to the melted wax with continuous stirring and heating until completely dispersed. Alternately, only the desired amount of the active ingredient is dispersed in the melted wax. The resulting granular-like particles are passed through a #20 mesh screen and placed in either a small PE bag or a 500 mL HDPE container. In the case of the melted wax containing only the active ingredient, the screened particles are blended with Lactose Fast Flo and Prosolv SMCC 90 for approximately 2 minutes in either a small PE bag or a 500 mL HDPE container. Approximately I g of each blend is mixed with the desired amounts of magnesium stearate and stearic acid, passed through a #30 mesh screen, returned to the blend, and mixed for approximately one minute. The final blend is compressed into tablets at weights of 225 mg (for tablets containing 100 mg active ingredient) and 630 mg or 675 mg (for tablets containing 100 mg active ingredient) using a conventional tablet press.

In another version of the fat-wax (hot melt) method, the desired amount of fat-wax, preferably, cetostearyl alcohol, is melted at approximately 70° C. in a mixer until the wax liquifies. The desired amounts of Lactose Fast Flo and Prosolv SMCC90 are blended for approximately 1 minute in a double lined PE bag and set aside. The desired amount of the active ingredient is added to the melted wax with continuous stirring and heating at approximately 70° C. until the active ingredient is completely dispersed. The blend of excipients is then added to the melted wax with stirring and maintaining heating between 40° C. and 60° C. until dispersion is complete. The resulting granular-like particles are cooled to ambient temperature, passed through a #20 mesh screen and placed in a double lined PE bag. The screened particles are then blended with stearic acid in a 4 quart V-shell for approximately 2 minutes at 25 rpm. Magnesium stearate is added to an equal amount of the stearic acid blend, blended in a small PE bag for approximately 1 minute, passed through a #20 mesh screen by hand, returned to the stearic acid blend and the final mixture is blended for 3 minutes at 25 rpm. The final blend was compressed into tablets at a weight of 630 mg or 675 mg (for tablets containing 300 mg of active ingredient) using a conventional tablet press.

The following Examples are provided as a guide to assist in the practice of the invention, and are not intended as a limitation on the scope of the invention.

### EXAMPLE 1

### 100 mg Controlled Release Tablet Formulation Hydrophilic Matrix System

A. A controlled release tablet formulation of the invention comprising 100 mg of the active ingredient in a hydrophilic matrix system is made by the direct compression method. In this formulation, the active ingredient, vernakalant hydrochloride, is mixed with Starch 1500 in a small polyethylene bag or a 500 mL HDPE container for approximately one minute, then passed through a #30 mesh screen. The screened mix is then transferred to its original polyethylene bag together with Prosolv SMCC 90, Lactose Fast Flo and Methocel K4M and mixed for 2 minutes. A portion (e.g., 1 g) of this blend is then mixed with magnesium stearate and stearic acid in a polyethylene bag, transferred back to the bulk blend via a #30 mesh screen and blended for 1 minute. Tablets may be compressed with a suitable punch. This formulation, hydrophilic formulation #100-1, is described in Table 7 below.

TABLE 7
100 MG HYDROPHILIC FORMULATION #100-1

| Ingredient | mg/tablet | % w/w | Wt.(g) |
|---|---|---|---|
| Active Ingredient | 100.00 | 33.33 | 5.00 |
| Starch 1500 | 15.00 | 5.00 | 0.75 |
| Prosolv SMCC 90 | 45.70 | 15.23 | 2.29 |
| Lactose Fast Flo | 91.30 | 30.43 | 4.57 |
| Methocel K4M | 45.00 | 15.00 | 2.25 |
| Stearic Acid | 1.50 | 0.50 | 0.08 |
| Magnesium stearate | 1.50 | 0.50 | 0.08 |
| Total Tablet Weight (mg) | 300.00 | 100.00 | 155.00 |

### EXAMPLE 2

### 100 mg Controlled Release Tablet Formulations Hydrophilic Matrix System

A. The following Table 8 provides for a controlled release tablet formulation of the invention comprising 100 mg of the active ingredient, vernakalant hydrochloride, in a hydrophilic matrix system. This formulation was prepared by the direct compression method disclosed herein using controlled-release grade Methocel K4M as the controlled release agent.

TABLE 8
100 MG HYDROPHILIC FORMULATION

| | Hydrophilic |
|---|---|
| | Formulation |
| | #100-2 |
| Ingredients | mg/tablet |
| Active Ingredient | 100.00 |
| Methocel K4M | 40.00 |
| Starch 1500 | 10.00 |
| Prosolv SMCC90 | 32.00 |
| Lactose Fast Flo | 40.00 |
| Stearic Acid | 1.50. |
| Magnesium Stearate | 1.50 |
| (Non-Bov) | |
| Total Tablet Weight (mg) | 225.00 |

B. Hydrophilic Formulation #100-2 was also prepared by the wet densification method described herein.

### EXAMPLE 3

300 mg Controlled Release Tablet Formulations Hydrophilic Matrix System

A. The following Table 9 provides for a controlled release tablet formulation of the invention comprising 300 mg of the active ingredient, vernakalant hydrochloride, in a hydrophilic matrix system. This formulation was prepared by compressing three times the weight of hydrophilic formulation #100-3.

TABLE 9
300 MG HYDROPHILIC FORMULATIONS

| | Hydrophilic |
|---|---|
| | Formulation |
| | #300-1 |
| Ingredients | mg/tablet |
| Active Ingredient | 300.00 |
| Methocel K4M | 120.00 |
| Starch 1500 | 30.00 |
| Prosolv SMCC90 | 96.00 |
| Lactose Fast Flo | 120.00 |
| Stearic Acid | 4.50 |
| Magnesium Stearate | 4.50 |
| (Non-Bov) | |
| Total Tablet Weight (mg) | 675.00 |

### EXAMPLE 4

### 300 mg Controlled Release Tablet Formulations Hydrophilic Matrix System

A. The following Table 10 provides for a controlled release tablet formulation of the invention comprising 300 mg of the active ingredient, vernakalant hydrochloride, in a hydrophilic matrix system. Hydrophilic formulation #300-2 was prepared by reducing the calculated tablet weight of 675 mg of hydrophilic formulation #300-1 to 630 mg by reducing the amount of Lactose Fast Flo and Prosolv SMCC 90.

TABLE 10
300 MG HYDROPHILIC FORMULATION

| | Hydrophilic |
|---|---|
| | Formulation |
| | #300-2 |
| Ingredients | mg/tablet |
| Active Ingredient | 300.00 |
| Methocel K4M | 120.00 |
| Starch 1500 | 30.00 |
| Prosolv SMCC90 | 90.00 |
| Lactose Fast Flo | 81.00 |
| Stearic Acid | 4.50 |
| Magnesium Stearate | 4.50 |
| (Non-Bov) | |
| Total Tablet Weight (mg) | 630.00 |

### EXAMPLE 5

### 100 mg Controlled Release Tablet Formulations Hydrophilic (Non-Cellulose) Matrix System

A. The following Table 11 provides for three controlled release tablet formulations of the invention comprising 100 mg of the active ingredient, vernakalant hydrochloride, in a hydrophilic (non-cellulose) matrix system. Hydrophilic (non-cellulose) formulations #100-1 and #100-3 were prepared by the direct compression method. Hydrophilic (non-cellulose) formulation #100-2 was prepared by the wet densification method described herein wherein the active ingredient and starch is mixed with water followed by drying and blending with the direct compression excipients. All three formulations had tablet weights of 225 mg.

TABLE 11
100 MG HYDROPHILIC (NON-CELLULOSE)
FORMULATIONS

| | Hydrophilic | Hydrophilic | Hydrophilic |
|---|---|---|---|
| | (Non-Cellulose) | (Non-Cellulose) | (Non-Cellulose) |
| | Formulation | Formulation | Formulation |
| | #100-1 | #100-2 | #100-3 |
| Ingredients | mg/tablet | mg/tablet | mg/tablet |
| Active | 100.00 | 100.00 | 100.00 |
| Ingredient | | | |
| Starch 1500 | — | 10.00 | 10.00 |
| Prosolv | 33.75 | 32.00 | 39.50 |
| SMCC90 | | | |
| Lactose | — | 46.25 | 50.00 |
| Fast Flo | | | |
| Polyethylene | 45.00 | — | — |
| Glycol | | | |
| Carbopol 971P | — | 33.75 | — |
| Polyox | — | — | 22.50 |
| WSR 1105 | | | |
| Anhydrous | 27.50 | — | — |
| Emcompress | | | |
| Eudragit | 15.75 | — | — |
| RS PO | | | |
| Stearic Acid | 1.50 | 1.50 | 1.50 |
| Magnesium | 1.50 | 1.50 | 1.50 |
| Stearate | | | |
| (Non-Bovine) | | | |
| Total Tablet | 225.00 | 225.00 | 225.00 |
| Weight (mg) | | | |

B. Hydrophilic (non-cellulose) formulation #100-2 was also prepared by the direct compression method described herein.

### EXAMPLE 6

### 300 mg Controlled Release Tablet Formulations Hydrophilic (Non-Cellulose) Matrix System

A. The following Table 12 provides for a controlled release tablet formulation of the invention comprising 300 mg of the active ingredient, vernakalant hydrochloride, in a hydrophilic (non-cellulose) matrix system. Hydrophilic (non-cellulose) formulation #300-1 was prepared by compressing three times the calculated weight of hydrophilic (non-cellulose) formulation #100-2 after reducing the calculated final tablet weight of 675 mg to 630 mg by reducing the amounts of Prosolv SMCC 90, Lactose Fast Flo and Carbopol 71G present in the final formulation.

TABLE 12
300 MG HYDROPHILIC (NON-CELLULOSE)
FORMULATION

| | Hydrophilic |
|---|---|
| | (Non-Cellulose) |
| | Formulation |
| | #300-1 |
| Ingredients | mg/tablet |
| Active Ingredient | 300.00 |
| Starch 1500 | 30.00 |
| Prosolv SMCC90 | 90.00 |
| Lactose Fast Flo | 105.00 |
| Carbopol 971P | 96.00 |
| Stearic Acid | 4.50 |
| Magnesium Stearate | 4.50 |
| (Non-Bovine) | |
| Total Tablet Weight (mg) | 630.00 |

### EXAMPLE 7

### 100 mg Controlled Release Formulations Hydrophobic Matrix System and Fat-Wax System

The following Table 13 provides for controlled release tablet formulations of the invention comprising 100 mg of the active ingredient, vernakalant hydrochloride, in a hydrophobic matrix system or in a fat-wax (hot-melt) system. These formulations were prepared by the methods disclosed herein. The hydrophilic matrix system formulation prepared in Example 1 is shown for comparison purposes only.

TABLE 13
100 MG CONTROLLED RELEASE FORMULATIONS

| | Hydrophobic | Fat-wax | Hydrophilic |
|---|---|---|---|
| | Formulation | Formulation | Formulation |
| | #100-1** | #100-1** | #100-1* |
| Ingredients | % w/w | % w/w | % w/w |
| Active Ingredient | 44.44 | 44.44 | 33.33 |
| Methocel K4M | — | — | 15.00 |
| Cetyl Alcohol | — | 22.22 | — |
| Ethylcellulose | 8.80 | — | — |
| Kollidon SR | 31.11 | — | — |
| Starch 1500 | — | — | 5.00 |
| Prosolv SMCC90 | — | 15.56 | 15.23 |
| Lactose | 14.22 | 16.44 | 30.43 |
| Stearic Acid | 0.67 | 0.67 | 0.50 |
| Magnesium Stearate | 0.67 | 0.67 | 0.50 |
| (Non-Bov) | | | |
| Total | 100.00 | 100.00 | 100.00 |

*Tablet weight: 300 mg
**Tablet weight: 225 mg

### EXAMPLE 8

### 100 mg Controlled Release Formulations Hydrophobic Matrix System

A. The following Table 14 provides for controlled release tablet formulations of the invention comprising 100 mg of the active ingredient, vernakalant hydrochloride, in a hydrophobic matrix system. Hydrophobic formulation #100-2 and hydrophobic formulation #100-3 were prepared using Kollidon SR and Ethylcellulose Standard 4 as the controlled release agents. Hydrophobic formulation #100-4 was prepared using Kollidon SR and Eudragit RS PO as the controlled release polymers. All three formulations were processed by the direct compression method disclosed herein.

TABLE 14
100 MG HYDROPHOBIC FORMULATIONS

| | Hydrophobic | Hydrophobic | Hydrophobic |
|---|---|---|---|
| | Formulation | Formulation | Formulation |
| | #100-2 | #100-3 | #100-4 |
| Ingredients | mg/tablet | mg/tablet | mg/tablet |
| Active Ingredient | 100.00 | 100.00 | 100.00 |
| Ethylcellulose | 20.00 | 20.00 | — |
| Kollidon SR | 70.00 | 70.00 | 45.00 |
| Starch 1500 | 32.00 | 32.00 | — |
| Anhydrous Emcompress | — | — | 32.00 |
| Eudragit RS PO | — | — | 45.00 |
| Prosolv SMCC90 | — | 15.56 | — |
| Stearic Acid | 1.50 | 1.50 | 1.50 |
| Magnesium Stearate | 1.50 | 1.50 | 1.50 |
| Total Tablet Weight (mg) | 225.00 | 225.00 | 225.00 |

B. The following Table 15 provides for a controlled release tablet formulation of the invention comprising 100 mg of the active ingredient, vernakalant hydrochloride, in a hydrophobic matrix system. This formulation, hydrophobic formulation #100-5, was prepared by the wet densification method.

TABLE 15
100 MG HYDROPHOBIC FORMULATION

| | Hydrophobic |
|---|---|
| | Formulation |
| | #100-5 |
| Ingredients | mg/tablet |
| Active Ingredient | 100.00 |
| Povidone K 29/32 | 11.00 |
| Kollidon SR | 60.00 |
| Anhydrous Emcompress | 31.00 |
| Eudragit RS PO | 20.00 |
| Stearic Acid | 1.50 |
| Magnesium Stearate | 1.50 |
| Total Tablet Weight (mg) | 225.00 |

### EXAMPLE 9

### 100 mg Controlled Release Formulations Hydrophilic/Hydrophobic Matrix System

A. The following Table 16 provides for a controlled release tablet formulation of the invention comprising 100 mg of the active ingredient, vernakalant hydrochloride, in a hydrophilic/hydrophobic matrix system. Hydrophilic/hydrophobic formulation #100-1 was prepared using Maltodextrin as the hydrophobic agent and Carbopol 71G as the hydrophilic controlled release agent. The formulation was prepared using the direct compression method disclosed herein.

TABLE 16
100 MG HYDROPHILIC/HYDROPHOBIC FORMULATION

| | Hydrophilic/Hydrophobic |
|---|---|
| | Formulation #100-1 |
| Ingredients | mg/tablet |
| Active Ingredient | 100.00 |
| Lactose Fast Flo | 39.00 |
| Carbopol 71G | 11.00 |
| Anhydrous Emcompress | 39.50 |
| Povidone K29/32 | 10.00 |
| Maltodextrin | 22.50 |
| Stearic Acid | 1.50 |
| Magnesium Stearate | 1.50 |
| Total Tablet Weight (mg) | 225.00 |

B. The following Table 17 provides for a controlled release tablet formulation of the invention comprising 100 mg of the active ingredient, vernakalant hydrochloride, in a hydrophilic/hydrophobic matrix system. Hydrophilic/hydrophobic formulation #100-2 was prepared using the wet densification method disclosed herein and Kollidon SR and Eudragit RS PO as its principal hydrophobic controlled release agent and Methocel K4M as its hydrophilic controlled release agent.

TABLE 17
100 MG HYDROPHILIC/HYDROPHOBIC FORMULATION

| | Hydrophilic/Hydrophobic |
|---|---|
| | Formulation #100-2 |
| Ingredients | mg/tablet |
| Active Ingredient | 100.00 |
| Methocel K4M | 11.00 |
| Kollidon SR | 50.00 |
| Anhydrous Emcompress | 30.00 |
| Povidone K29/32 | 11.00 |
| Eudragit RS PO | 20.00 |
| Stearic Acid | 1.50 |
| Magnesium Stearate | 1.50 |
| Total Tablet Weight (mg) | 225.00 |

### EXAMPLE 10

### 300 mg Controlled Release Formulations Hydrophilic/Hydrophobic Matrix System

The following Table 18 provides for a controlled release tablet formulation of the invention comprising 300 mg of the active ingredient, vernakalant hydrochloride, in a hydrophilic/hydrophobic matrix system. Hydrophilic/hydrophobic formulation #300-1 was prepared by compressing three times the weight of hydrophilic/hydrophobic formulation #100-2 (the calculated final tablet weight of 675 mg was reduced to 630 mg by reducing and adjusting the amounts of Methocel K4M, Povidone K29/32, Kollidon SR, Eudragit RS PO and Anhydrous Emcompress present in the formulation).

TABLE 18
300 MG HYDROPHILIC/HYDROPHOBIC FORMULATION

| | Hydrophilic/Hydrophobic |
|---|---|
| | Formulation #300-1 |
| Ingredients | mg/tablet |
| Active Ingredient | 300.00 |
| Methocel K4M | 30.00 |
| Kollidon SR | 135.00 |
| Anhydrous Emcompress | 81.00 |
| Povidone K29/32 | 30.00 |
| Eudragit RS PO | 45.00 |
| Stearic Acid | 4.50 |
| Magnesium Stearate | 4.50 |
| Total Tablet Weight (mg) | 630.00 |

### EXAMPLE 11

### 100 mg Controlled Release Formulations Fat-Wax System

A. The following Table 19 provides for three controlled release tablet formulations of the invention comprising 100 mg of the active ingredient, vernakalant hydrochloride, in a fat-wax system. All three formulations were prepared by the fat-wax method disclosed herein where all the ingredients, except magnesium stearate and stearic acid, were dispersed in the wax, i.e., cetostearyl alcohol.

TABLE 19
100 MG FAT-WAX FORMULATIONS

| | Fat-wax | Fat-wax | Fat-wax |
|---|---|---|---|
| | Formulation | Formulation | Formulation |
| | #100-2 | #100-3 | #100-4 |
| Ingredients | mg/tablet | mg/tablet | mg/tablet |
| Active Ingredient | 100.00 | 100.00 | 100.00 |
| Prosolv SMCC 90 | 50.00 | 35.00 | 35.00 |
| Lactose Fast Flo | 37.00 | 37.00 | 37.00 |
| Cetostearyl Alcohol | 35.00 | 50.00 | 50.00 |
| Stearic Acid | 1.50 | 1.50 | 1.50 |
| Magnesium Stearate | 1.50 | 1.50 | 1.50 |
| Total Tablet Weight (mg) | 225.00 | 225.00 | 225.00 |

B. Fat-wax formulation #100-3 and #100-4 were also prepared by the fat-wax method described herein wherein only the active ingredient is dispersed in the fat-wax.

C. The following Table 20 provides for a controlled release tablet formulation of the invention comprising 100 mg of the active ingredient, vernakalant hydrochloride, in a fat-wax system. The formulation was prepared by the fat-wax method wherein only the active ingredient is dispersed in the fat-wax, i.e., cetyl alcohol.

TABLE 20
100 MG FAT-WAX FORMULATIONS

| | Fat-wax |
|---|---|
| | Formulation |
| | #100-5 |
| Ingredients | mg/tablet |
| Active Ingredient | 100.00 |
| Prosolv SMCC 90 | 29.75 |
| Lactose Fast Flo | 36.00 |
| Cetyl Alcohol | 56.25 |
| Stearic Acid | 1.50 |
| Magnesium Stearate | 1.50 |
| Total Tablet Weight (mg) | 225.00 |

### EXAMPLE 12

### 300 mg Controlled Release Formulations Fat-Wax System

The following Table 21 provides for a controlled release tablet formulation of the invention comprising 300 mg of the active ingredient, vernakalant hydrochloride, in a fat-wax system. This formulation was prepared by methods disclosed herein.

TABLE 21
300 MG FAT-WAX FORMULATIONS

| | Fat-wax |
|---|---|
| | Formulation |
| | #300-1 |
| Ingredients | mg/tablet |
| Active Ingredient | 300.00 |
| Prosolv SMCC 90 | 105.00 |
| Lactose Fast Flo | 111.00 |
| Cetostearyl Alcohol | 150.00 |
| Stearic Acid | 4.50 |
| Magnesium Stearate | 4.50 |
| Total Tablet Weight (mg) | 675.00 |

### EXAMPLE 13

### 100 mg Immediate Release Formulation

An immediate release tablet formulation comprising 100 mg of the active ingredient, vernakalant hydrochloride, was prepared for comparison purposes only by the direct compression method disclosed herein. The formulation was blended in small PE bags and subsequently compressed manually on a single punch bench tablet press with an appropriate tablet punch. The active ingredient was mixed with Starch 1500 in a small PE bag and then passed through a # 30 mesh screen. The screened mix was then transferred to its original polyethylene bag along with Prosolv SMCC90, Lactose Fast Flo and Explotab and mixed for 2 minutes. A portion (e.g., 1 g) of this blend was then mixed with magnesium stearate and stearic acid in a PE bag, transferred back to the bulk blend via a #30 mesh screen and blended for 1 minute. Tablets were compressed with a suitable punch on a single punch press to obtain a tablet hardness of 7-12 KN. The formulation is described in Table 22 below.

TABLE 22
100 MG IMMEDIATE RELEASE TABLET FORMULATION

| Ingredient | mg/tab | % w/w | Wt.(g) |
|---|---|---|---|
| Ion Channel Modulating | 100.00 | 33.33 | 5.00 |
| Compound | | | |
| Explotab (Sodium Starch | 3.00 | 1.00 | 0.15 |
| Glycolate) | | | |
| Lactose Fast Flo | 117.50 | 39.17 | 5.88 |
| Magnesium Stearate | 1.50 | 0.50 | 0.08 |
| Prosolv SMCC90 | 60.00 | 20.00 | 3.00 |
| Starch 1500 | 15.00 | 5.00 | 0.75 |
| Stearic Acid | 3.00 | 1.00 | 0.15 |
| Total Tablet Weight | 300.00 | 100.00 | 15.00 |

### In vitro Release Profile of the Formulations of the Invention

The in vitro release profile of the formulations of the invention may be empirically determined by examining the dissolution of the tablet formulations over time. A USP approved method for dissolution or release test can be used to measure the rate of release in vitro (USP 24; NF 19 (2000) pp. 1941-1951). For example, a weighed tablet containing the active ingredient is added to a measured volume of a solution containing 0.9% NaCl in water, where the solution volume will be such that the active ingredient concentration after release is less than 20% of saturation. The mixture is maintained at 37° C. and stirred or shaken slowly to maintain the tablet in suspension. The release of the dissolved active ingredient as a function of time may then be followed by various methods known in the art, such as spectrophotometrically, HPLC, mass spectroscopy, and the like, until the solution concentration becomes constant or until greater than 90% of the active ingredient has been released.

The following Example is provided as a guide to assist in the practice of the invention, and is not intended as a limitation on the scope of the invention.

### EXAMPLE 14

### In-vitro Dissolution of Controlled Release Tablet Formulations of the Invention

A. The following Table 23 provides the mean dissolution release percentages of selected controlled tablet formulations of the invention comprising 100 mg of the active ingredient. The dissolved percentages indicated are mean values based on the number of tablets tested for each formulation.

TABLE 23
MEAN DISSOLUTION % RELEASE OF 100 MG CONTROLLED
RELEASE FORMULATIONS

| | Formulations | | | | | |
|---|---|---|---|---|---|---|
| | | Hydrophilic | | | | |
| | | (Non- | | | | Hydrophilic/ |
| | Hydrophilic | Celluose) | Hydrophobic | Fat-wax | Fat-wax | Hydrophobic |
| | #100-2 | #100-2 | #100-5 | #100-4 | #100-5 | #100-2 |
| % Dissolved | 23 | 22 | 18 | 29 | 30 | 16 |
| after 0.5 hrs | | | | | | |
| % Dissolved | 32 | 29 | 29 | 42 | 41 | 24 |
| after 1 hour | | | | | | |
| % Dissolved | 45 | 39 | 45 | 58 | 57 | 36 |
| after 2 hours | | | | | | |
| % Dissolved | 61 | 54 | 67 | 78 | 85 | 53 |
| after 4 hours | | | | | | |
| % Dissolved | 71 | 67 | 82 | 91 | 95 | 66 |
| after 6 hours | | | | | | |
| % Dissolved | 78 | 78 | 88 | 98 | 97 | 74 |
| after 8 hours | | | | | | |
| % Dissolved | 83 | 87 | 92 | 102 | 98 | 81 |
| after 10 hours | | | | | | |
| % Dissolved | 86 | 93 | 94 | 103 | 99 | 85 |
| after 12 hours | | | | | | |
| # of tablets | 6 | 6 | 6 | 6 | 3 | 6 |
| tested | | | | | | |
| Active | 22.6 | 1.6 | 16.7 | 7.6 | 1.6 | 12.7 |
| Ingredient | | | | | | |
| Remaining at | | | | | | |
| 12 hours (%) | | | | | | |

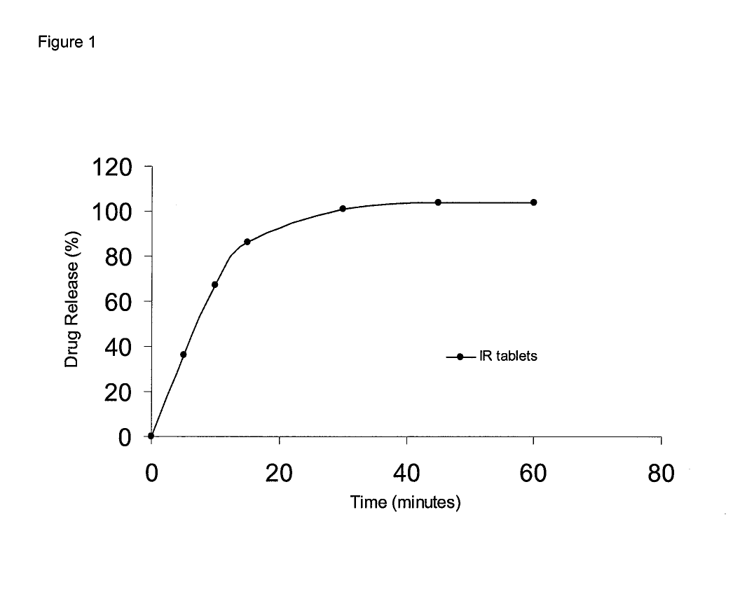

For comparison purposes only, FIG. 1 shows a release profile (percent cumulative release over time) for the immediate release tablet formulation comprising 100 mg of the active ingredient (as described above in Example 13). More than 80% of the active ingredient in the immediate release tablet formulation dissolved by fifteen minutes.

B. The following Table 24 provides the dissolution release percentages of selected controlled tablet formulations of the invention comprising 300 mg of the active ingredient.

TABLE 24
MEAN DISSOLUTION % RELEASE OF 300 MG CONTROLLED
RELEASE FORMULATIONS

| | Formulations | | | |
|---|---|---|---|---|
| | | Hydrophilic | | |
| | | (Non- | | Hydrophilic/ |
| | Hydrophilic | Celluose) | Fat-wax | Hydrophobic |
| | #300-2 | #300-1 | #300-1 | #300-1 |
| % Dissolved | 18 | 16 | 30 | 26 |
| after 0.5 hrs | | | | |
| % Dissolved | 27 | 21 | 41 | 36 |
| after 1 hour | | | | |
| % Dissolved | 41 | 28 | 57 | 47 |
| after 2 hours | | | | |
| % Dissolved | 61 | 41 | 75 | 63 |
| after 4 hours | | | | |
| % Dissolved | 75 | 53 | 87 | 74 |
| after 6 hours | | | | |
| % Dissolved | 85 | 66 | 95 | 82 |
| after 8 hours | | | | |
| % Dissolved | 92 | 78 | 99 | 89 |
| after 10 hours | | | | |
| % Dissolved | 97 | 91 | 101 | 94 |
| after 12 hours | | | | |

### In vivo Pharmacokinetic Profiles of the Formulations of the Invention

The in vivo pharmacokinetic profiles of the formulations of the invention were determined as follows. Formulations of the invention were administered to dogs in a controlled experiment to determine pharmacokinetic profile of each formulation tested. A single controlled release tablet formulation of the invention was orally administered to each group of dogs. Blood samples were collected via the jugular or cephalic vein at predose (0), 15, 30, 60, 90, 120, 240, 360, 480, 600 and 1440 minutes after administration or at predose (0), 30, 60, 90, 120, 240, 360, 480, 600, 720 and 1440 minutes after administration. Concentration levels of the active ingredient in the plasma samples at each timepoint was determined using standard methods known to one skilled in the art. The concentration levels were plotted on a standard pharmacokinetic curve (time in minutes versus concentration in ng/mL) and the area under the curve extrapolated to infinity (AUC_{inf}), the Cₘₐₓ (peak blood plasma concentration level of the active ingredient) and Tₘₐₓ (time after administration of the formulation when peak plasma concentration level occurs) were calculated. In general, a controlled release formulation should provide a broader pharmacokinetic curve while minimizing the Cₘₐₓ when compared to the pharmacokinetic curve of an immediate release formulation. In other words, a large ratio of AUC_{inf}/Cₘₐₓ is desired for each controlled release formulation of the invention. As noted below in Example 15 and 16, the controlled release tablet formulations of the invention demonstrated the ability to release the active ingredient into the blood over a longer period of time than the corresponding immediate release formulation.

### EXAMPLE 15

### In vivo Pharmacokinetic Profiles of Formulations of the Invention

A. The following Table 25 presents the plasma concentrations of the active ingredient, vernakalant hydrochloride, in dogs that received the immediate release tablet formulation comprising 100 mg of the active ingredient, vernakalant hydrochloride, as set forth above in Example 13; a hydrophilic controlled release tablet formulation of the invention comprising 100 mg of the active ingredient, (i.e., hydrophilic formulation #100-2); a hydrophilic controlled release tablet formulation of the invention comprising 300 mg of the active ingredient (i.e., hydrophilic formulation #300-1); a fat-wax controlled release tablet formulation of the invention comprising 100 mg of the active ingredient (i.e., fat-wax formulation #100-3); and a hydrophobic controlled release tablet formulation of the invention comprising 100 mg of the active ingredient (i.e., hydrophobic formulation #100-3). Concentrations are given as pg/mL and are an average obtained from n=3 dogs unless otherwise indicated.

TABLE 25
PLASMA CONCENTRATIONS OF ACTIVE INGREDIENT IN DOGS
AFTER ORAL ADMINISTRATION OF VARIOUS FORMULATIONS
OF THE INVENTION

| | Formulations | | | | |
|---|---|---|---|---|---|
| Time | Immediate | Hydrophilic | Hydrophilic | Fat-wax | Hydrophobic |
| (min) | Release | #100-2 | #300-1 | #100-3 | #100-3 |
| 0 | ND | ND | ND | ND | ND |
| 15 | 0.398* | ND | 0.078 | 0.224* | 0.072* |
| 30 | 0.770* | 0.047** | 0.152 | 0.445 | 0.135 |
| 60 | 0.522* | 0.077 | 0.194 | 0.596 | 0.201 |
| 90 | 0.303 | 0.211 | 0.230 | 0.599 | 0.244 |
| 120 | 0.259 | 0.255 | 0.413 | 0.448 | 0.236 |
| 240 | 0.094 | 0.375 | 0.986 | 0.113 | 0.197 |
| 360 | 0.046 | 0.239 | 0.795 | 0.118** | 0.100 |
| 480 | 0.029 | 0.147 | 0.581 | 0.077** | 0.069 |
| 600 | 0.025 | 0.077 | 0.236 | 0.045** | 0.048* |
| 1440 | ND | ND | ND | ND | ND |

ND = No detection,
*n = 2,
**n = 1

From the above concentration averages, the area under the curve (AUC_{inf}), Tₘₐₓ and Cₘₐₓ were calculated and their ratio determined (AUC_{inf}/Cₘₐₓ), as shown in Table 26 below. All four controlled release formulations had later Tₘₐₓ than the immediate release formulation. For the ratio of AUC_{inf}/Cₘₐₓ, the immediate release formulation had the lowest ratio out the five formulations, while the hydrophilic and the fat-wax formulations had the best ratios. In addition, a dose-dependent increase in AUC_{inf} was observed for the concentrations of hydrophilic formulation #300-1 as compared to hydrophilic formulation #100-2.

TABLE 26
PHARMACOKINETIC PARAMETERS

| | Formulations | | | | |
|---|---|---|---|---|---|
| | Imme- | | | | |
| | diate | Hydrophilic | Hydrophilic | Fat-wax | Hydrophobic |
| | Release | #100-2 | #300-1 | #100-3 | #100-3 |
| Cₘₐₓ | 0.770 | 0.375 | 0.986 | 0.599 | 0.244 |
| (μg/mL) | | | | | |
| Tₘₐₓ | 30 | 240 | 240 | 90 | 90 |
| (minutes) | | | | | |
| AUC_{inf} | 96 | 143 | 407 | 134 | 94 |
| (μg/ML/ | | | | | |
| minutes) | | | | | |
| AUC_{inf}/ | 125 | 381 | 413 | 223 | 387 |
| Cₘₐₓ | | | | | |

As shown above, all four controlled release tablet formulations have later Tₘₐₓ than the immediate release table formulations and all four had broader pharmacokinetic curves while minimizing the Cₘₐₓ.

B. The following Table 27 presents the plasma concentrations of the active ingredient, vernakalant hydrochloride, in dogs that received a hydrophilic controlled release tablet formulation of the invention comprising 300 mg of the active ingredient, (i.e., hydrophilic formulation #300-2); a fat-wax controlled release tablet formulation of the invention comprising 300 mg of the active ingredient (i.e., fat-wax formulation #300-1); a hydrophilic/hydrophobic controlled release tablet formulation of the invention comprising 300 mg of the active ingredient (i.e., hydrophobic formulation #300-1), and a hydrophilic (non-cellulose) tablet formulation of the invention comprising 300 mg of the active ingredient (i.e., hydrophilic (non-cellulose) formulation #300-1). Concentrations are given as μg/mL and are an average obtained from n=3 dogs unless otherwise indicated.

TABLE 27
PLASMA CONCENTRATIONS OF ACTIVE INGREDIENT IN DOGS
AFTER ORAL ADMINISTRATION OF VARIOUS FORMULATIONS
OF THE INVENTION

| | Formulations | | | |
|---|---|---|---|---|
| | | | Hydrophilic/ | Hydrophilic |
| | Hydrophilic | Fat-wax | Hydrophobic | (non-cellulose) |
| Time (min) | #300-2 | #300-1 | #300-1 | #300-1 |
| 0 | ND | ND | ND | ND |
| 30 | 0.321* | 0.240 | 0.058* | 0.513 |
| 60 | 0.560 | 0.696* | 0.244 | 1.976 |
| 90 | 0.992 | 1.031 | 1.096 | 2.945 |
| 120 | 0.981 | 0.973 | 1.217 | 2.421 |
| 240 | 1.516 | 0.833 | 1.475 | 0.692 |
| 360 | 0.785 | 0.720 | 0.468 | 0.321 |
| 480 | 0.329 | 0.600 | 0.272 | 0.239 |
| 600 | 0.209 | 0.539 | 0.147 | 0.218 |
| 720 | 0.171 | 0.352* | 0.100 | 0.141 |
| 1440 | 0.042 | 0.056** | 0.015* | 0.026* |

ND = No detection,
*n = 2,
**n = 1

From the above concentration averages, the area under the curve (AUC_{inf}), Tₘₐₓ and Cₘₐₓ were calculated and their ratio determined (AUC_{inf}/Cₘₐₓ), as shown in Table 28 below. For the ratio of AUC_{inf}/Cₘₐₓ, the hydrophilic formulation #300-2 and the fat-wax formulation #300-1 had the best ratios.

TABLE 28
PHARMACOKINETIC PARAMETERS

| | Formulations | | | |
|---|---|---|---|---|
| | | | Hydrophilic/ | Hydrophilic |
| | Hydrophilic | Fat-wax | Hydrophobic | (non-cellulose) |
| | #300-2 | #300-1 | #300-1 | #300-1 |
| Cₘₐₓ (μg/mL) | 1.516 | 1.031 | 1.475 | 2.945 |
| Tₘₐₓ (minutes) | 240 | 90 | 240 | 90 |
| AUC_{inf} | 578 | 645 | 469 | 600 |
| (μg/ML/minutes) | | | | |
| AUC_{inf}/Cₘₐₓ | 381 | 626 | 318 | 204 |

Compared to a comparable immediate release table formulation, all four formulations have the later Tₘₐₓ and broader pharmacokinetic curves while minimizing the Cₘₐₓ.

### In vivo Pharmacokinetic Profiles of Controlled Release Formulations of the Invention Administered to Humans

Hydrophilic formulation #300-2 and Fat Wax formulation #300-2 were each administered as one dose (300 mg of active ingredient) to six healthy male and female subjects (six subjects per formulation). Blood was drawn at pre-dose (0 hours), 0.5, 1, 1.5, 2, 3, 4, 6, 8, 10, 12, 16 and 24 hours post dose. The median pharmacokinetic parameters of each formulation are shown in the following Table 29:

TABLE 29
PHARMACOKINETIC PARAMETERS OF 300 MG ACTIVE
INGREDIENT

| | Comparative | Comparative |
|---|---|---|
| | Hydrophilic | Fat Wax |
| | #300-2 | #300-1 |
| Cₘₐₓ (ng/mL) | 290 | 296 |
| Tₘₐₓ (hours) | 2.0 | 1.75 |
| AUC_{0-inf} | 2029 | 1984 |
| (ng/mL/hour) | | |

Based on the above results, hydrophilic formulation #300-2 was administered as a double dose (600 mg of active ingredient) to six healthy male and female subjects. Blood was drawn at pre-dose (0 hours), 0.5, 1, 1.5, 2, 3, 4, 6, 8, 10, 12, 16 and 24 hours post dose. The median pharmacokinetic parameters are shown in the following Table 30:

TABLE 30
PHARMACOKINETIC PARAMETERS OF 600 MG ACTIVE
INGREDIENT

| | Comparative |
|---|---|
| | Hydrophilic |
| | #300-2 (X2) |
| Cₘₐₓ (ng/mL) | 706 |
| Tₘₐₓ (hours) | 2.0 |
| AUC_{0-inf} | 5307 |
| (ng/mL/hour) | |

Prevention of Recurrence of Atrial Fibrillation/Atrial Flutter

The following study was conducted to evaluate, inter alia, the efficacy of formulations of the invention in human subjects with sustained atrial fibrillation (atrial fibrillation of longer than 72 hours and less than 6 months duration).

Subjects were administered a formulation of the invention on Day 1 and were monitored for the first 3 days of dosing. Subjects who were still in atrial fibrillation on the third day of dosing were electrically converted to sinus rhythm. Subjects who converted to sinus rhythm without intervention (other than study medication) or who were successfully electrocardioverted continued with study medication for a total of 28 days of study treatment administration.

A total of 221 subjects were enrolled in the study, 75 subjects received placebo, 71 subjects received twice daily one capsule containing one controlled release tablet formulation of the invention (300 mg active ingredient b.i.d.), hydrophilic formulation #300-2, 75 subjects received twice daily one capsule containing two controlled release tablet formulations of the invention (600 mg active ingredient b.i.d.), hydrophilic formulation #300-2. The majority of the study subjects were male (61.4%) and Caucasian (100%), with a mean ag of 64±10 years (range 32-83 years). A total of 171 subjects were converted to sinus rhythm by Day 3 of the study and continued to received study medication through Day 28.

The time to first documented recurrence of symptomatic sustained atrial fibrillation or atrial flutter was longer in subjects receiving the active ingredient than subjects receiving placebo. 43.1% of placebo subjects were in sinus rhythymm on Day 28 compared to 61.6% of subjects treated with 300 mg. active ingredient b.i.d. and 62.4% of subjects treated with 600 mg active ingredient b.i.d.

This study demonstrated the ability of hydrophilic formulation #300-2 to reduce the short term recurrence of atrial fibrillation.

All of the U.S. patents, U.S. patent application publications, U.S. patent applications, foreign patents, foreign patent applications and non-patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

1. A controlled release tablet formulation comprising a therapeutically effective amount of ion channel modulating compound, or a pharmaceutically acceptable salt thereof and one or more pharmaceutically acceptable excipients.

2. The controlled release tablet formulation of claim 1 wherein the ion channel modulating compound or pharmaceutically acceptable salt thereof is vemakalant hydrochloride.

3. The controlled release tablet formulation of claim 2 wherein at least one of the pharmaceutically acceptable excipients is a hydrophilic matrix system polymer selected from the group consisting of carbomer, maltodextrin, hydroxyethyl cellulose, hydroxypropyl cellulose, hydroxypropyl methyl cellulose, and polyoxoacetate.

4. The controlled release tablet formulation of claim 3 wherein the hydrophilic matrix system polymer is hydroxypropyl methyl cellulose.

5. The controlled release tablet formulation of claim 4 comprising:
300 mg of vernakalant hydrochloride;
120 mg of hydroxypropyl methyl cellulose;
30 mg preglatinized starch;
90 mg silicified microcrystalline cellulose;
81 mg of lactose monohydrate;
4.5 mg stearic acid; and
4.5 mg magnesium stearate.

6. The controlled release tablet formulation of claim 2 wherein at least one of the pharmaceutically acceptable excipients is a erodable retardant selected from the group consisting of cetyl alcohol or cetostearyl alcohol.

7. The controlled release tablet formulation of claim 6 wherein the erodable retardant is cetostearyl alcohol.

8. The controlled release tablet formulation of claim 7 comprising:
300 mg of vernakalant hydrochloride;
150 mg cetostearyl alcohol;
105 mg silicified microcrystalline cellulose;
111 mg of lactose monohydrate;
4.5 mg stearic acid; and
4.5 mg magnesium stearate.

9. A method of preventing the recurrence of an arrhythmia in a mammal that has previously undergone one or more arrhythmia, wherein the method comprises administering to the mammal in need thereof a therapeutically effective amount of a controlled release formulation comprising a therapeutically effective amount of ion channel modulating compound, or a pharmaceutically acceptable salt thereof and one or more pharmaceutically acceptable excipients.

10. The method of claim 9 wherein the arrhythmia is atrial fibrillation.

11. The method of claim 10 wherein the mammal is a human.

12. The method of claim 11 wherein the ion channel modulating compound or pharmaceutically acceptable salt thereof of the controlled release formulation is vemakalant hydrochloride.

13. The method of claim 12 wherein at least one of the pharmaceutically acceptable excipients of the controlled release formulation is a hydrophilic matrix system polymer selected from the group consisting of carbomer, maltodextrin, hydroxyethyl cellulose, hydroxypropyl cellulose, hydroxypropyl methyl cellulose, and polyoxoacetate.

14. The method of claim 13 wherein the hydrophilic matrix system polymer is hydroxypropyl methyl cellulose.

15. The method of claim 14 wherein the controlled release formulation comprises:
300 mg of vernakalant hydrochloride;
120 mg of hydroxypropyl methyl cellulose;
30 mg preglatinized starch;
90 mg silicified microcrystalline cellulose;
81 mg of lactose monohydrate;
4.5 mg stearic acid; and
4.5 mg magnesium stearate.

16. The method of claim 12 wherein wherein at least one of the pharmaceutically acceptable excipients of the controlled release formulation is a erodable retardant selected from the group consisting of cetyl alcohol or cetostearyl alcohol.

17. The method of claim 16 wherein the wherein the erodable retardant is cetostearyl alcohol.

18. The method of claim 17 wherein the controlled release formulation comprises:
300 mg of vernakalant hydrochloride;
150 mg cetostearyl alcohol;
105 mg silicified microcrystalline cellulose;
111 mg of lactose monohydrate;
4.5 mg stearic acid; and
4.5 mg magnesium stearate.
